SEQUENCE LISTING (2 sequences)

SEQ ID NO: 1 - DNA, 348 bp, Artificial Sequence
  Gene Sequence of BMP-2 Stem Cells
atgcaggctaaacacaaacagcgtaaacgtctgaaatcttcttgcaaacgtcacccgctgtacgttgacttctctgacgttggttggaacgactggatcgttgctccgccgggttaccacgctttctactgccacggtgaatgcccgttcccgctggctgaccacctgaactctaccaaccacgctatcgttcagaccctggttaactctgttaactctaaaatcccgaaagcttgctgcgttccgaccgaactgtctgctatctctatgctgtacctggacgaaaacgaaaaagttgttctgaaaaactaccaggacatggttgttgaaggttgcggttgccgttaa

SEQ ID NO: 2 - DNA, 7052 bp, Artificial Sequence
  Sequence of Vector pET-30a-BMP-2 Stem Cell-PDI
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaatgttgacaattaatcatccggctcgtataatgtgtggaattgtgagcggataacaattcccctctagaaataattttgtttaactttaagaaggagatatacatatgcaccatcatcatcatcattcttctggtcaggctaaacacaaacagcgtaaacgtctgaaatcttcttgcaaacgtcacccgctgtacgttgacttctctgacgttggttggaacgactggatcgttgctccgccgggttaccacgctttctactgccacggtgaatgcccgttcccgctggctgaccacctgaactctaccaaccacgctatcgttcagaccctggttaactctgttaactctaaaatcccgaaagcttgctgcgttccgaccgaactgtctgctatctctatgctgtacctggacgaaaacgaaaaagttgttctgaaaaactaccaggacatggttgttgaaggttgcggttgccgtctggaagttctgttccaggggccccctgaagactccgctgtcgttaagttggccaccgactccttcaatgagtacattcagtcgcacgacttggtgcttgcggagttttttgctccatggtgtggccactgtaagaacatggctcctgaatacgttaaagccgccgagactttagttgagaaaaacattaccttggcccagatcgactgtactgaaaaccaggatctgtgtatggaacacaacattccagggttcccaagcttgaagattttcaaaaacagcgatgttaacaactcgatcgattacgagggacctagaactgccgaggccattgtccaattcatgatcaagcaaagccaaccggctgtcgccgttgttgctgatctaccagcttaccttgctaacgagacttttgtcactccagttatcgtccaatccggtaagattgacgccgacttcaacgccaccttttactccatggccaacaaacacttcaacgactacgactttgtctccgctgaaaacgcagacgatgatttcaagctttctatttacttgccctccgccatggacgagcctgtagtatacaacggtaagaaagccgatatcgctgacgctgatgtttttgaaaaatggttgcaagtggaagccttgccctactttggtgaaatcgacggttccgttttcgcccaatacgtcgaaagcggtttgcctttgggttacttattctacaatgacgaggaagaattggaagaatacaagcctctctttaccgagttggccaaaaagaacagaggtctaatgaactttgttagcatcgatgccagaaaattcggcagacacgccggcaacttgaacatgaaggaacaattccctctatttgccatccacgacatgactgaagacttgaagtacggtttgcctcaactctctgaagaggcgtttgacgaattgagcgacaagatcgtgttggagtctaaggctattgaatctttggttaaggacttcttgaaaggtgatgcctccccaatcgtgaagtcccaagagatcttcgagaaccaagattcctctgtcttccaattggtcggtaagaaccatgacgaaatcgtcaacgacccaaagaaggacgttcttgttttgtactatgccccatggtgtggtcactgtaagagattggccccaacttaccaagaactagctgatacctacgccaacgccacatccgacgttttgattgctaaactagaccacactgaaaacgatgtcagaggcgtcgtaattgaaggttacccaacaatcgtcttatacccaggtggtaagaagtccgaatctgttgtgtaccaaggttcaagatccttggactctttattcgacttcatcaaggaaaacggtcacttcgacgtcgacggtaaggccttgtacgaagaagcccaggaaaaagctgctgaggaagccgatgctgacgctgaattggctgacgaagaagatgccattcacgatgaattgtaaagcaataactagcataaccccttggggcctctaaacgggtcttgaggggttttttgctgaaaggaggaactatatccggat

(19) United States
(12) Patent Application Publication
(10) Pub. No.: US 2020/0109369 A1
(43) Pub. Date: Apr. 9, 2020

(54) INNOVATIVE PREPARATION PROCESS FOR BMP-2 STEM CELLS

(71) Applicant: Jinyang Yi, Shenzhen (CN)

(72) Inventor: Jinyang Yi, Shenzhen (CN)

(21) Appl. No.: 16/565,528

(22) Filed: Sep. 10, 2019

(51) Int. Cl. C12N 5/0775; C12N 15/85; C12N 15/74; C12N 15/70; C07K 1/22; C07K 1/36

(52) U.S. Cl. CPC C12N 5/0667; C12N 15/85; C12N 15/74; C12N 2015/8518; C07K 1/22; C07K 1/36; C12N 15/70

(57) ABSTRACT

The present invention discloses an innovative preparation process for recombinant human fat cell peptide (BMP-2 stem cells for short), comprising the following steps: gene synthesis of BMP-2 stem cells, specific expression system construction of mature peptide, expression induction, separation, purification and amplification. The method disclosed by the present invention is simple and flexible; has simple product separation and purification process, high purity, high activity and low production cost; has the functions of preventing clinical virus, resisting cell aging, and promoting wound healing and repair in epidermis, dermis and mucosa; and has broad application prospects in the fields such as medical care, cosmetology and health care.

### TECHNICAL FIELD

The present invention belongs to the field of biotechnology, and specifically relates to an innovative process and preparation method for BMP-2 stem cells with the effects of wound scar repair, wrinkle removal and skin aging resistance.

### BACKGROUND

In the process of skin wound healing, with the production of a large amount of collagen, fat cells are largely lost, leading to scar formation; the process of skin aging is accompanied by the loss of fat cells, leading to the appearance of wrinkles which are the “natural enemies” of every woman. In order to keep the skin young and smooth, women spend a lot of money on different skin care products and cosmetics, but these cosmetics usually cover up wrinkles from the surface only, and do not fundamentally eliminate wrinkles. Moreover, fat cell loss is not only a natural result of human aging, but also a common complication of female reproductive system immunity declination and HIV infection.

A recent study by Prof. George Cotsarelis of the University of Pennsylvania shows that (Plikus, Maksim V., et al. “Regeneration of fat cells from myofibroblasts during wound healing.” Science 355.6326 (2017): 748-752.) hair follicles hold the key to keeping wounds healing and skin smooth without scars, because they release an important signaling molecule called “bone morphogenetic protein” (BMP), and BMP will direct those cells that form scars in the wound—myofibroblasts—to transform into fat cells. This groundbreaking new discovery will not only bring exciting anti-viral and anti-aging treatments to humans, but also pave a way for inactivating various viral infections, resisting cellular aging, and achieving wound healing without scars. One of the reasons for permanent wrinkles on the faces of the elderly is the lack of fat cells. As scientists find ways to regenerate fat cells, wrinkles on people's faces will become a thing of the past. Treatment with BMP-2 stem cells can reverse human crow's feet and wrinkles and make the skin look younger, and BMP-2 stem cells can better repair the wound, restore the damaged cells to be smooth, firm and elastic, reduce scar formation and prevent pigmentation.

It is generally believed that a myofibroblast cannot become another cell, but the study by Prof. George Cotsarelis shows that with the inductive effect of BMP, myofibroblasts can be effectively and stably transformed into fat cells to manipulate the wound healing process and make the process goes to skin regeneration instead of scarring. According to a report on the website of the Daily Telegraph on Jan. 8, 2017: the method for fat cell regeneration not only improves cell filling, but also effectively prevents wound scar formation, reduces skin roughness and wrinkles, delays cell aging, promotes the reversed growth of epidermal cells of a human body, and makes people's skin younger, smoother and more elastic, which will bring more new treatments for treating HIV, resisting aging and wrinkles, reducing wound scar formation and promoting wound healing.

BMP is a class of acidic glycoproteins, which belongs to the transforming growth factor TGF-β superfamily and was first discovered in 1965. So far, more than 20 BMP members have been discovered. Among which, BMP-2 stem cells are the most widely studied, are approved by the US Food and Drug Administration (FDA) and the European Medicines Agency (EMEA) for clinical use, and have broad application prospects. BMP-2 stem cells can not only inhibit and inactivate a variety of viruses, but also cause the aged cells of a human body to reversely differentiate into young cells, in order to achieve the purpose of rapidly healing wounds. However, natural BMP-2 stein cells are rare and have complicated separation process, low purity, poor repeatability, difficult quality control, high cost and easy deactivation, and are therefore not suitable for industrial production and are severely limited in clinical application. Using genetic recombination technology can not only achieve large-scale production, but also ensure the quality of products, which has excellent market development prospects.

At present, BMP-2 stem cells can be expressed by both eukaryotic and prokaryotic expression systems. The eukaryotic expression system uses mammalian cells, and the expression products thereof can be glycosylated, have better post-translational modifications, and are relatively more active. Most of the current foreign products are prepared by using the eukaryotic expression system. However, the eukaryotic expression system has a low expression quantity, and therefore the recovery and purification of the expression products are difficult, resulting in high production cost. The current foreign BMP products are very expensive, and the clinical popularization thereof is very difficult. Compared with the eukaryotic expression system, E. coli expression system has mature technology, high expression quantity and low cost, and has become a hot spot technology for the production of BMP-2 stem cells by gene recombination. The prokaryotic expression system has the advantages of strong tolerance to culture environment, easy control, stable product, low production cost, etc., and is therefore more conducive to clinical popularization. However, the main problem is that it is difficult to process and fold correctly to form active proteins, so that most of the work stays at laboratory level and cannot be industrialized.

### SUMMARY

The present invention provides a kind of BMP-2 stem cells with a function of inducing fat cell regeneration; and the gene of the BMP-2 stem cells is derived from human genome.

The present invention is implemented by the following technical solution:

The present invention performs codon optimization to the sequence of fat cells according to a human genome sequence and the codon usage frequency of E. coli, synthesizes the gene base sequence of BMP-2 stem cells by whole gene recombination, adds a 6×HIS tag sequence to the 5′-terminus of the gene sequence of BMP-2 stem cells to achieve protein purification, and increases a molecular chaperone protein PDI derived from Saccharomyces cerevisiae at the 3′-terminus to promote the soluble expression thereof. An expression vector pET-30a(+) is used to express the gene in E. coli, and separate and purify the encoded protein. Sequencing analysis shows that the open reading frame of the gene contains 342 bases. The sequence is as shown in SEQ ID NO. 1, and the encoded BMP-2 stem cells have a molecular weight of 12.5 kDa.

The present invention obtains a coded gene from human genome, and the encoded protein thereof can induce myofibroblasts to effectively and stably transform into BMP-2 stem cells, which can compensate for the loss of fat cells in the formation process of wound scars, wrinkles and skin aging. The products of the present invention can be used for wound healing, scar repair, skin aging resistance, wrinkle removal, etc., thereby achieving the effect of eternal youth.

Compared with the prior art, by using the gene recombinant expression method of the present invention, including obtaining the gene of BMP-2 stem cells by whole gene synthesis and constructing a high-efficiency expression vector by a Trc promoter and a molecular chaperone protein, the BMP-2 stem cells are maintained in a correctly folded state with solubility and high activity, and a higher protease activity than an inclusion body is maintained; the addition of a 6×HIS tag to the sequence is conducive to the high-efficiency purification of the protein, which greatly simplifies the purification step, and thus the BMP-2 stem cells prepared thereby have high purity, high activity, stable product quality and low production cost, has the function of promoting clinical wound healing, repair and aging resistance in epidermis, dermis and mucosa, and has great application value in the fields of medical care, cosmetology and health care.

### DESCRIPTION OF DRAWINGS

FIG. 1 shows the construction of an expression vector pET-30a-BMP-2 stem cell-PDI map containing the gene of BMP-2 stem cells;

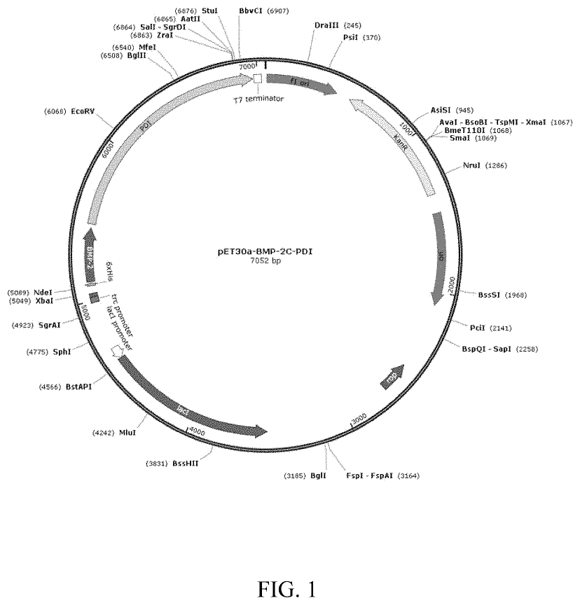

FIG. 2 is an SDS-PAGE analysis of the induced expression of BMP-2 stem cell protein (containing PDI).

### The Present Invention Takes the Following Specific Measures:

I. Gene Synthesis of BMP-2 Stem Cells and Construction of Expression Vector

1. Gene Synthesis of BMP-2 Stem Cells by Multiple Rounds of PCR Amplification

Taking a plasmid pET-30a(+)-PDI (in which a T7 promoter has been replaced by a Trc promoter) containing a molecular chaperone PDI derived from Saccharomyces cerevisiae as a template, using primers P1/P2, P1-2/P2-2, P1-3/P2-3, P1-4/P2-4 and P1-5/P2-5 (containing 20 bp of left and right homology arms), and performing multiple rounds of PCR amplification respectively. The PCR reaction system and cycle settings are as follows:

| | | |
|---|---|---|
| PrimerSTAR Max | 10 | μL |
| DNA Polymerase | | |
| P1 | 1 | μL |
| P2 | 1 | μL |
| DNA template | 0.1 | μL |
| Total volume | 20 | μL |

The primers used for amplification are as follows:

| | |
|---|---|
| P1 | CAGGACATGGTTGTTGAAGGTTGCGGTTGCCGTGAAGTTCTGTT |
| | CCAGGGGCCC |
| P1-2 | TGGACGAAAACGAAAAAGTTGTTCTGAAAAACTACCAGGACATG |
| | GTTGTTGAAGGTTG |
| P1-3 | TCCGACCGAACTGTCTGCTATCTCTATGCTGTACCTGGACGAAA |
| | ACGAAAAAGTTGTT |
| P1-4 | TCTGTTAACTCTAAAATCCCGAAAGCTTGCTGCGTTCCGACCGA |
| | ACTGTCTGCTATCT |
| P1-5 | ACTCTACCAACCACGCTATCGTTCAGACCCTGGTTAACTCTGTT |
| | AACTCTAAAATCCCG |
| P2 | TTTCAGACGTTTACGCTGTTTGTGTTTAGCCTGACCAGAAGAAT |
| | GATGATGATGATG |
| P2-2 | AGAAGTCAACGTACAGCGGGTGACGTTTGCAAGAAGATTTCAGA |
| | CGTTTACGCTGTTTG |
| P2-3 | GGCGGAGCAACGATCCAGTCGTTCCAACCAACGTCAGAGAAGTC |
| | AACGTACAGCGGG |
| P2-4 | GAACGGGCATTCACCGTGGCAGTAGAAAGCGTGGTAACCCGGCG |
| | GAGCAACGATCCAG |
| P2-5 | CGATAGCGTGGTTGGTAGAGTTCAGGTGGTCAGCCAGCGGGAAC |
| | GGGCATTCACCGTGG |

Mixing well and performing PCR amplification according to the following procedure: performing pre-denaturation at 98° C. for 5 min to enter a cycle, performing denaturation at 98° C. for 10 s, performing annealing at 55° C. for 15 s, performing extension at 72° C. for 1 min, and continuing to perform extension at 72° C. for 5 min after 7 cycles (30 cycles during the last round of amplification). After the reaction is completed, using DpnI to digest the possibly residual plasmid template.

2. Transformation of Digestion Product into E. Coli DH5α Competent Cells

(1) Putting the competent cells on ice and making the cells melt slowly;

(2) Adding the above digestion product and PCR product, mixing well with the competent cells, and keeping the mixture in an ice bath for 30 min;

(3) After a heat shock at 42° C. for 90 s, centrifuging and transferring the products to ice immediately, and let the products stand for 2 min;

(4) Adding 1 mL of LB culture medium and incubating at 37° C. for 1 h;

(5) After centrifugation, applying an appropriate amount of bacteria solution to an LB plate containing kanamycin and performing inverted culture at 37° C. overnight for 12 h.

3. Gene Sequencing of BMP-2 Stem Cells

Picking an E. coli transformant which is initially identified by PCR and has the same size as a target fragment, naming the transformant as pET-30a-BMP-2 stem cell-PDI, putting the transformant in a LB culture medium for overnight culture, and extracting the plasmid. Performing DNA sequencing to the above transformant, and the nucleotide sequence of the gene of BMP-2 stem cells obtained is as shown in SEQ ID No. 1.

II. Prokaryotic Expression of Gene of BMP-2 Stem Cells and Separation & Purification of Expression Protein

1. Gene Expression of BMP-2 Stem Cells in E. Coli Transetta (DE3)

(1) Extracting the recombinant plasmid pET-30a-BMP-2 stem cell-PDI and transforming the plasmid into E. coli Transetta (DE3).

(2) Picking a monoclonal colony for culture, inoculating the colony into a fresh LB culture medium (containing kanamycin) by a proportion of 1%, continuing to culture the colony until the OD600 is about 1, adding IPTG until the final concentration is 0.1 mmol/L, and performing induced culture at 30° C. and 200 r/min overnight for 12 h.

(3) SDS-PAGE Analysis of Expression Products

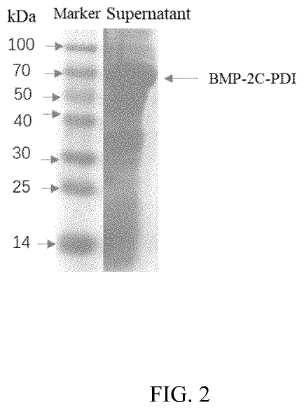

Adding 1 mL of the above induced fermentation product into a 1.5 mL centrifuge tube, removing the supernatant by centrifugation, adding 0.2 mL of PBS solution, performing cell disruption, obtaining supernatant and sediment after centrifugation, mixing the supernatant and sediment respectively with 50 μL of 5×SDS-PAGE loading buffer, keeping the mixture in a boiling water bath for 10 min, and detecting the expression of BMP-2 stem cells in E. coli by SDS-PAGE. The results are as shown in FIG. 2. After induction by IPTG, the BMP-2 stem cells (containing PDI) are expressed in a large amount and are mainly expressed in a soluble form in the supernatant.

2. Purification of Recombinant Expression BMP-2 Stem Cells

In a BMP-2 stem cell solution with a high concentration, cutting with Prescission Protease at 4° C. to release BMP-2 stem cells, and adjusting the PH to 8.0. Preparing column-loading buffer:

| | |
|---|---|
| MCAC-15 | MCAC-1000 |
| 20 mM/L Tris-HCl pH 10 | 20 mM/L Tris-HCl pH 10 |
| 0.5 M/L NaCl | 0.5M/L NaCl |
| 10% (v/v) Glycerine | 10% (v/v) Glycerine |
| 15 mM/L Imidazole | 1M/L Imidazole |

Washing a nickel ion affinity column (Ni-NTA His-Bind Resin) filler with MCAC-15 solution, and then co-culture the filler and the BMP-2 stem cell solution; after gently shaking for 30 min, applying the BMP-2 stem cell solution to the column, eluting the hybrid protein with MCAC-50 (obtained by diluting MCAC-1000), and finally eluting the target protein with MCAC-250. Dialyzing the obtained product overnight for 12 h to obtain a BMP-2 stem cell fine product with a high purity.

SEQ ID NO. 1
Gene Sequence of BMP-2 Stem Cells
ATGCAGGCTAAACACAAACAGCGTAAACGTCTGAAATCTTCTTGCAAACG
TCACCCGCTGTACGTTGACTTCTCTGACGTTGGTTGGAACGACTGGATCG
TTGCTCCGCCGGGTTACCACGCTTTCTACTGCCACGGTGAATGCCCGTTC
CCGCTGGCTGACCACCTGAACTCTACCAACCACGCTATCGTTCAGACCCT
GGTTAACTCTGTTAACTCTAAAATCCCGAAAGCTTGCTGCGTTCCGACCG
AACTGTCTGCTATCTCTATGCTGTACCTGGACGAAAACGAAAAAGTTGTT
CTGAAAAACTACCAGGACATGGTTGTTGAAGGTTGCGGTTGCCGTTAA
SEQ ID NO. 2
Sequence of Vector pET-30a-BMP-2 Stem Cell-PDI
tggcgaatgggacgcgccctgtagcggcgcattaagcgcggcgggtgtgg
tggttacgcgcagcgtgaccgctacacttgccagcgccctagcgcccgct
cctttcgattatcccttcctttctcgccacgttcgccggctttccccgtc
aagctctaaatcgggggctccctttagggttccgatttagtgattacggc
acctcgaccccaaaaaacttgattagggtgatggttcacgtagtgggcca
tcgccctgatagacggtttttcgccctttgacgttggagtccacgttctt
taatagtggactcttgttccaaactggaacaacactcaaccctatctcgg
tctattcttttgatttataagggattttgccgatttcggcctattggtta
aaaaatgagctgatttaacaaaaatttaacgcgaattttaacaaaatatt
aacgtttacaatttcaggtggcacttttcggggaaatgtgcgcggaaccc
ctatttgtttatttttctaaatacattcaaatatgtatccgctcatgaat
taattcttagaaaaactcatcgagcatcaaatgaaactgcaatttattca
tatcaggattatcaataccatatttttgaaaaagccgtttctgtaatgaa
ggagaaaactcaccgaggcagttccataggatggcaagatcctggtatcg
gtctgcgattccgactcgtccaacatcaatacaacctattaatttcccct
cgtcaaaaataaggttatcaagtgagaaatcaccatgagtgacgactgaa
tccggtgagaatggcaaaagtttatgcatttctttccagacttgttcaac
aggccagccattacgctcgtcatcaaaatcactcgcatcaaccaaaccgt
tattcattcgtgattgcgcctgagcgagacgaaatacgcgatcgctgtta
aaaggacaattacaaacaggaatcgaatgcaaccggcgcaggaacactgc
cagcgcatcaacaatattttcacctgaatcaggatattcttctaatacct
ggaatgctgttttcccggggatcgcagtggtgagtaaccatgcatcatca
ggagtacggataaaatgcttgatggtcggaagaggcataaattccgtcag
ccagtttagtctgaccatctcatctgtaacatcattggcaacgctacatt
gccatgtttcagaaacaactctggcgcatcgggcttcccatacaatcgat
agattgtcgcacctgattgcccgacattatcgcgagcccatttataccca
tataaatcagcatccatgttggaatttaatcgcggcctagagcaagacgt
ttcccgttgaatatggctcataacaccccttgtattactgtttatgtaag
cagacagttttattgttcatgaccaaaatccataacgtgagttttcgttc
cactgagcgtcagaccccgtagaaaagatcaaaggatcttcttgagatcc
tttttttctgcgcgtaatctgctgcttgcaaacaaaaaaaccaccgctac
cagcggtggtttgtttgccggatcaagagctaccaactctttttccgaag
gtaactggcttcagcagagcgcagataccaaatactgtccttctagtgta
gccgtagttaggccaccacttcaagaactctgtagcaccgcctacatacc
tcgctctgctaatcctgttaccagtggctgctgccagtggcgataagtcg
tgtcttaccgggaggactcaagacgatagttaccggataaggcgcagcgg
tcgggctgaacggggggttcgtgcacacagcccagcttggagcgaacgac
ctacaccgaactgagatacctacagcgtgagctatgagaaagcgccacgc
ttcccgaagggagaaaggcggacaggtatccggtaagcggcagggtcgga
acaggagagcgcacgagggagcttccagggggaaacgcctggtatcttta
tagtcctgtcgggtttcgccacctctgacttgagcgtcgatttttgtgat
gctcgtcaggggggcggagcctatggaaaaacgccagcaacgcggccttt
ttacggttcctggccttttgctggccttttgctcacatgactttcctgcg
ttatcccctgattctgtggataaccgtattaccgcctttgagtgagctga
taccgctcgccgcagccgaacgaccgagcgcagcgagtcagtgagcgagg
aagcggaagagcgcctgatgcggtattttctccttacgcatctgtgcggt
atttcacaccgcatatatggtgcactctcagtacaatctgctctgatgcc
gcatagttaagccagtatacactccgctatcgctacgtgactgggtcatg
gctgcgccccgacacccgccaacacccgctgacgcgccctgacgggcttg
tctgctcccggcatccgcttacagacaagctgtgaccgtctccgggagct
gcatgtgtcagaggttttcaccgtcatcaccgaaacgcgcgaggcagctg
cggtaaagctcatcagcgtggtcgtgaagcgattcacagatgtctgcctg
acatccgcgtccagctcgttgagtttctccagaagcgttaatgtctggct
tctgataaagcgggccatgttaagggcggttttttcctgtttggtcactg
atgcctccgtgtaagggggatttctgttcatgggggtaatgataccgatg
aaacgagagaggatgctcacgatacgggttactgatgatgaacatgcccg
gttactggaacgttgtgagggtaaacaactggcggtatggatgcggcggg
accagagaaaaatcactcagggtcaatgccagcgcttcgttaatacagat
gtaggtgttccacagggtagccagcagcatcctgcgatgcagatccggaa
cataatggtgcagggcgctgacttccgcgtttccagactttacgaaacac
ggaaaccgaagaccattcatgttgttgctcaggtcgcagacgttttgcag
cagcagtcgcttcacgttcgctcgcgtatcggtgattcattctgctaacc
agtaaggcaaccccgccagcctagccgggtcctcaacgacaggagcacga
tcatgcgcacccgtggggccgccatgccggcgataatggcctgcttctcg
ccgaaacgtttggtggcgggaccagtgacgaaggcttgagcgagggcgtg
caagattccgaataccgcaagcgacaggccgatcatcgtcgcgctccagc
gaaagcggtcctcgccgaaaatgacccagagcgctgccggcacctgtcct
acgagttgcatgataaagaagacagtcataagtgcggcgacgatagtcat
gccccgcgcccaccggaaggagctgactgggttgaaggctctcaagggca
tcggtcgagatcccggtgcctaatgagtgagctaacttacattaattgcg
ttgcgctcactgcccgctttccagtcgggaaacctgtcgtgccagctgca
ttaatgaatcggccaacgcgcggggagaggcggtttgcgtattgggcgcc
agggtggtttttcttttcaccagtgagacgggcaacagctgattgccctt
caccgcctggccctgagagagttgcagcaagcggtccacgctggtttgcc
ccagcaggcgaaaatcctgtttgatggtggttaacggcgggatataacat
gagctgtcttcggtatcgtcgtatcccactaccgagatgtccgcaccaac
gcgcagcccggactcggtaatggcgcgcattgcgcccagcgccatctgat
cgttggcaaccagcatcgcagtgggaacgatgccctcattcagcatttgc
atggtttgttgaaaaccggacatggcactccagtcgccttcccgttccgc
tatcggctgaatttgattgcgagtgagatatttatgccagccagccagac
gcagacgcgccgagacagaacttaatgggcccgctaacagcgcgatttgc
tggtgacccaatgcgaccagatgctccacgcccagtcgcgtaccgtatca
tgggagaaaataatactgttgatgggtgtctggtcagagacatcaagaaa
taacgccggaacattagtgcaggcagcttccacagcaatggcatcctggt
catccagcggatagttaatgatcagcccactgacgcgagcgcgagaagat
tgtgcaccgccgctttacaggcttcgacgccgcttcgttctaccatcgac
accaccacgctggcacccagttgatcggcgcgagatttaatcgccgcgac
aatttgcgacggcgcgtgcagggccagactggaggtggcaacgccaatca
gcaacgactgtttgcccgccagttgttgtgccacgcggttgggaatgtaa
ttcagctccgccatcgccgcttccactttttcccgcgttttcgcagaaac
gtggctggcctggttcaccacgcgggaaacggtctgataagagacaccgg
catactctgcgacatcgtataacgttactggtttcacattcaccaccctg
aattgactctcttccgggcgctatcatgccataccgcgaaaggattgcgc
cattcgatggtgtccgggatctcgacgctctcccttatgcgactcagcat
taggaagcagcccagtagtaggttgaggccgttgagcaccgccgccgcaa
ggaatggtgcatgcaaggagatggcgcccaacagtcccccggccacgggg
cctgccaccatacccacgccgaaacaagcgctcatgagcccgaagtggcg
agcccgatcttccccatcggtgatgtcggcgatataggcgccagcaaccg
cacctgtggcgccggtgatgccggccacgatgcgtccggcgtagaggatc
gagatcgatctcgatcccgcgaaatGTTGACAATTAATCATCCGGCTCGT
ATAATGTGTggaattgtgagcggataacaattcccactagaaataatttt
gtttaactttaagaaggagatatacatatgcaccatcatcatcatcattc
ttctggTCAGGCTAAACACAAACAGCGTAAACGTCTGAAATCTTCTTGCA
AACGTCACCCGCTGTACGTTGACTTCTCTGACGTTGGTTGGAACGACTGG
ATCGTTGCTCCGCCGGGTTACCACGCTTTCTACTGCCACGGTGAATGCCC
GTTCCCGCTGGCTGACCACCTGAACTCTACCAACCACGCTATCGTTCAGA
CCCTGGTTAACTCTGTTAACTCTAAAATCCCGAAAGCTTGCTGCGTTCCG
ACCGAACTGTCTGCTATCTCTATGCTGTACCTGGACGAAAACGAAAAAGT
TGTTCTGAAAAACTACCAGGACATGGTTGTTGAAGGTTGCGGTTGCCGTC
TGGAAGTTCTGTTCCAGGGGCCCCCTGAAGACTCCGCTGTCGTTAAGTTG
GCCACCGACTCCTTCAATGAGTACATTCAGTCGCACGACTTGGTGCTTGC
GGAGTTTTTTGCTCCATGGTGTGGCCACTGTAAGAACATGGCTCCTGAAT
ACGTTAAAGCCGCCGAGACTTTAGTTGAGAAAAACATTACCTTGGCCCAG
ATCGACTGTACTGAAAACCAGGATCTGTGTATGGAACACAACATTCCAGG
GTTCCCAAGCTTGAAGATTTTCAAAAACAGCGATGTTAACAACTCGATCG
ATTACGAGGGACCTAGAACTGCCGAGGCCATTGTCCAATTCATGATCAAG
CAAAGCCAACCGGCTGTCGCCGTTGTTGCTGATCTACCAGCTTACCTTGC
TAACGAGACTTTTGTCACTCCAGTTATCGTCCAATCCGGTAAGATTGACG
CCGACTTCAACGCCACCTTTTACTCCATGGCCAACAAACACTTCAACGAC
TACGACTTTGTCTCCGCTGAAAACGCAGACGATGATTTCAAGCTTTCTAT
TTACTTGCCCTCCGCCATGGACGAGCCTGTAGTATACAACGGTAAGAAAG
CCGATATCGCTGACGCTGATGTTTTTGAAAAATGGTTGCAAGTGGAAGCC
TTGCCCTACTTTGGTGAAATCGACGGTTCCGTTTTCGCCCAATACGTCGA
AAGCGGTTTGCCTTTGGGTTACTTATTCTACAATGACGAGGAAGAATTGG
AAGAATACAAGCCTCTCTTTACCGAGTTGGCCAAAAAGAACAGAGGTCTA
ATGAACTTTGTTAGCATCGATGCCAGAAAATTCGGCAGACACGCCGGCAA
CTTGAACATGAAGGAACAATTCCCTCTATTTGCCATCCACGACATGACTG
AAGACTTGAAGTACGGTTTGCCTCAACTCTCTGAAGAGGCGTTTGACGAA
TTGAGCGACAAGATCGTGTTGGAGTCTAAGGCTATTGAATCTTTGGTTAA
GGACTTCTTGAAAGGTGATGCCTCCCCAATCGTGAAGTCCCAAGAGATCT
TCGAGAACCAAGATTCCTCTGTCTTCCAATTGGTCGGTAAGAACCATGAC
GAAATCGTCAACGACCCAAAGAAGGACGTTCTTGTTTTGTACTATGCCCC
ATGGTGTGGTCACTGTAAGAGATTGGCCCCAACTTACCAAGAACTAGCTG
ATACCTACGCCAACGCCACATCCGACGTTTTGATTGCTAAACTAGACCAC
ACTGAAAACGATCTTCAGAGGCGTCGTAATTGAAGGTTACCCAACAATCG
TCTTATACCCAGGTGGTAAGAAGTCCGAATCTGTTGTGTACCAAGGTTCA
AGATCCTTGGACTCTTTATTCGACTTCATCAAGGAAAACGGTCACTTCGA
CGTCGACGGTAAGGCCTTGTACGAAGAAGCCCAGGAAAAAGCTGCTGAGG
AAGCCGATGCTGACGCTGAATTGGCTGACGAAGAAGATGCCATTCACGAT
GAATTGTAAagcaataactagcataaccccttggggcctctaaacgggtc
ttgaggggttttttgctgaaaggaggaactatatccggat

1. An innovative preparation process for BMP-2 stem cells, characterized in comprising the following steps in sequence:
(1) constructing an expression vector while rapidly synthesizing a gene by multiplex PCR;
(2) introducing an expression plasmid into a host bacteria to construct an expression engineering bacteria;
(3) fermenting the engineering bacteria and inducing expression at 30° C.; and
(4) performing enzymatic cleavage to expression products to release BMP-2 stem cells, and purifying the expression products by a nickel column to obtain a BMP-2 stem cell fine product.

2. The innovative preparation process of claim 1, characterized in that in step (1), a series of primers are designed, the full length of a target gene is obtained by multiple rounds of PCR amplification, a vector with homology arms (containing the target gene) is obtained at the same time, self-ligation is achieved by PCR to construct a circular DNA sequence containing a gap, and complete BMP-2 stem cells of the expression vector are obtained by intracellular repair through transformed E. coli.

3. The innovative preparation process of claim 1, characterized in that the gene of BMP-2 stem cells in the expression vector is placed below a Trc promoter.

4. The innovative preparation process of claim 1, characterized in that the C-terminus of the gene of BMP-2 stem cells in the expression vector is ligated with a molecular chaperone PDI gene that promotes folding.

5. The innovative preparation process of claim 1, characterized in that the N-terminus of the gene of BMP-2 stem cells in the expression vector is ligated with a 6×HIS tag used for separation.

6. The innovative preparation process of claim 1, characterized in that the constructed optimal expression vector is introduced into E. coli Transetta (DE3) to construct an expression engineering bacteria.

7. The innovative preparation process of claim 1, characterized in that through induction at 30° C., most products are expressed in a soluble form, BMP-2 stem cells are released by low-temperature enzymatic cleavage, and a fine product is obtained after separation and purification by a nickel column.

8. The innovative preparation process of claim 1, characterized in that BMP-2 stem cells have the functions of preventing clinical virus, resisting cell aging, and promoting wound healing and repair in epidermis, dermis and mucosa; and have broad application prospects in the fields such as medical care, cosmetology and health care.
